(19) United States
(12) Patent Application Publication
(10) Pub. No.: US 2026/0000837 A1
(43) Pub. Date: Jan. 1, 2026

(54) DELIVERY DEVICES

(71) Applicant: Boston Scientific Scimed, Inc., Maple Grove, MN (US)

(72) Inventors: Benjamin Cleveland, Bellingham, MA (US); Yen-Hao Hsu, Shrewsbury, MA (US)

(73) Assignee: Boston Scientific Scimed, Inc., Maple Grove, MN (US)

(21) Appl. No.: 19/250,706

(22) Filed: Jun. 26, 2025

Related U.S. Application Data
(60) Provisional application No. 63/665,144, filed on Jun. 27, 2024.

(51) Int. Cl. A61M 5/31; A61M 39/10

(52) U.S. Cl. CPC A61M 5/3129; A61M 39/10; A61M 2202/0468

(57) ABSTRACT

A delivery device comprising a first syringe comprising a first plunger and a first syringe barrel that terminates in a first connector; a second syringe comprising a second plunger and a second syringe barrel that terminates in a second connector; a Y-connector coupled to the first syringe and the second syringe; and an injection member coupled to the Y-connector, where the injection member includes a plurality of fluid ejection ports that extend through a sidewall of the injection member.

### CROSS-REFERENCE TO RELATED APPLICATIONS

This application claims the benefit of U.S. Provisional Patent Application Ser. No. 63/665,144 filed on Jun. 27, 2024, the disclosure of which is incorporated herein by reference.

### FIELD

The present disclosure relates to improved delivery devices, systems, and methods. The improved delivery devices, systems, and methods may be employed to deliver or inject hydrogels, among other possible materials. Hydrogels are useful, for example, in various medical applications.

### BACKGROUND

Hydrogels with shear-thinning properties are known to undergo a reversible gel-sol transition upon the application of shear stress. J. Pushpamalar, et al., “Development of a polysaccharide-based hydrogel drug delivery system (DDS): An update.” Gels, 2021, 7(4), p. 153. Shear-thinning hydrogels are increasingly used in drug delivery systems as a result of their ability to conform to the shape of an injection cavity, which maximizes contact with targeted tissue for localized drug delivery. Id. Hydrogels with shear-thinning properties have been reported to provide smooth injection without injection needle clogging (e.g., partial or complete flow restriction), with the hydrogels returning to their original properties once mechanical load (shear stress) is removed. M. H. Chen, et al., “Methods to assess shear-thinning hydrogels for application as injectable biomaterials.” ACS Biomater. Sci. Eng. 2017, 3, 3146-3160. Hydrogels assembled by physical crosslinking of polyvinyl alcohol (PVA) and polyvinyl pyrrolidone (PVP) and containing doxorubicin have been noted to be able to deform under high shear and subsequently retain their original shape upon the removal of the high shear, demonstrating both shear-thinning and self-healing properties. N. K. Prasad, et al., “Discerning the self-healing, shear-thinning characteristics and therapeutic efficacy of hydrogel drug carriers migrating through constricted microchannel resembling blood microcapillary,” Colloids Surf. A Physiochem. Eng. Asp. 2021, 626, 127070. FIG. 1 on page 5 of J. Pushpamalar, et al., schematically illustrates the shear-thinning and self-healing properties of a doxorubicin-loaded poly (vinyl alcohol)/poly (vinyl pyrrolidone) hydrogel. A shear-thinning hydrogel containing gelatin and laponite for localized drug delivery and further containing chitosan and poly (N-isopropylacrylamide-co-acrylic acid) particles to render the hydrogel pH-responsive has also been reported. S. Gharaie, et al., “Smart shear-thinning hydrogels as injectable drug delivery systems.” Polymers, 2018, 10(12), p. 1317.

SpaceOAR® is a rapid crosslinking hydrogel that polymerizes in vivo and is based on a multi-arm polyethylene glycol (PEG) polymer functionalized with succinimidyl glutarate as activated end groups which further react with trilysine to form crosslinks. This product has been used clinically in prostate cancer therapy.

### BRIEF SUMMARY

A first example is a delivery device comprising: a first syringe comprising a first plunger and a first syringe barrel that terminates in a first connector; a second syringe comprising a second plunger and a second syringe barrel that terminates in a second connector; a Y-connector coupled to the first syringe and the second syringe; and an injection member coupled to the Y-connector, wherein the injection member includes a plurality of fluid injection ports that extends through a sidewall of the injection member.

Alternatively or additionally to any of the examples herein, in another example, wherein the injection member is a needle or a tube.

Alternatively or additionally to any of the examples herein, in another example, wherein the plurality of fluid injection ports is located at a distal end region of the injection member.

Alternatively or additionally to any of the examples herein, in another example, wherein the injection member includes an injection lumen extending substantially longitudinally therethrough and that is in fluid communication with a common lumen in the Y-connector, wherein the injection lumen is in fluid communication with at least the plurality of fluid injection ports.

Alternatively or additionally to any of the examples herein, in another example, wherein a distal tip of the injection member is beveled.

Alternatively or additionally to any of the examples herein, in another example, wherein: a distal tip of the injection member is substantially rounded; and the injection member is movably disposed within a lumen of a sheath, the sheath having a beveled distal tip.

Alternatively or additionally to any of the examples herein, in another example, wherein the injection lumen terminates at a proximal end of the substantially rounded distal tip.

Alternatively or additionally to any of the examples herein, in another example, wherein each fluid ejection port of the plurality of injection ports is substantially the same shape and the same size.

Alternatively or additionally to any of the examples herein, in another example, wherein each fluid ejection port of the plurality of fluid ejection ports is a substantially circular fluid ejection port.

Alternatively or additionally to any of the examples herein, in another example, wherein the sidewall is an annular sidewall, and wherein the plurality of fluid injection ports is located circumferentially about the annular sidewall.

Alternatively or additionally to any of the examples herein, in another example, wherein the plurality of fluid injection ports is located in a distal end region of the injection member.

Alternatively or additionally to any of the examples herein, in another example, wherein: the first plunger is movable between a first position and a second position independent of a position of the second plunger; and the second plunger is movable between a first position and a second position independent of a position of the first plunger.

Alternatively or additionally to any of the examples herein, in another example, wherein the plurality of fluid injection ports is configured as a plurality of sets of fluid injection ports that are spaced apart along a longitudinal axis of the delivery device, and wherein each set of fluid injection ports includes a plurality of fluid injection ports extending circumferentially about the annular sidewall of the injection member.

Alternatively or additionally to any of the examples herein, in another example, wherein: wherein the first syringe contains a first solution comprising a viscosity-modified buffered precursor solution including a buffered precursor solution and a viscosity modifier; and the second syringe contains a second solution comprising a buffered accelerant solution.

Alternatively or additionally to any of the examples herein, in another example, wherein each fluid ejection port has a diameter that is configured to permit the viscosity modifier to pass therethrough, and wherein the viscosity modifier comprises polylactic acid-co-glycolic acid (PLGA), polylactic acid (PLA), or a hydrophilic polymer.

Another example is a delivery device comprising: a first syringe comprising a first plunger and a first syringe barrel that terminates in a first connector, wherein the first syringe contains a first composition comprising a viscosity-modified buffered precursor solution including a buffered precursor solution and a viscosity modifier; and a second syringe comprising a second plunger and a second syringe barrel that terminates in a second connector, wherein the second syringe contains a buffered accelerant solution; a Y-connector coupled to the first syringe and the second syringe; and an injection member coupled to the Y-connector, wherein the injection member includes: an annular sidewall with an injection lumen extending therethrough; and a plurality of fluid injection ports that are in fluid communication with the injection lumen and that extend through the annular sidewall; and wherein the delivery device is configured to sequentially inject the viscosity-modified buffered precursor solution followed by the buffered accelerant solution.

Alternatively or additionally to any of the examples herein, in another example, wherein the viscosity modifier comprises polylactic acid-co-glycolic acid (PLGA), polylactic acid (PLA), or a hydrophilic polymer.

Alternatively or additionally to any of the examples herein, in another example, wherein the viscosity modifier is present in an amount in a range from about 0.5 to about 10 weight percent based on a total weight of the viscosity-modified buffered precursor solution.

Alternatively or additionally to any of the examples herein, in another example, wherein the viscosity-modified buffered precursor solution has a viscosity that is at least five percent higher than a viscosity of the buffered precursor solution.

Yet another example is a kit comprising: a first reservoir containing a first composition comprising a viscosity-modified buffered precursor solution including a buffered precursor solution and a viscosity modifier; a second reservoir containing a second composition comprising a buffered accelerant solution; and a delivery device comprising: a Y-connector coupled to the first reservoir and the second reservoir; and an injection member coupled to the Y-connector, wherein the injection member includes: an annular sidewall with an injection lumen extending therethrough; and a plurality of fluid injection ports that are in fluid communication with the injection lumen and that extend through the annular sidewall; and wherein the delivery device is configured to sequentially inject the viscosity-modified buffered precursor solution followed by the buffered accelerant solution.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

FIG. 1 schematically illustrates an example of delivery device;

FIG. 2A illustrates a simplified representation of a target site and a simplified view of the example of delivery device of FIG. 1 with a first plunger in a first position and a second plunger in a first position;

FIG. 2B illustrates a simplified representation of a target site and simplified view of the example delivery device of FIG. 1 with a first plunger in a second position and a second plunger in a first position;

FIG. 2C illustrates a simplified representation of a target site and a simplified view of the example of delivery device of FIG. 1 with a first plunger in a second position and a second plunger in a second position;

FIG. 3 illustrates an enlarged view of a portion of the injection member of the delivery device of FIG. 1;

FIG. 4A illustrates an enlarged view of a portion of the injection member of the delivery device of FIG. 1 with the injection member in a first position; and

FIG. 4B illustrates an enlarged view of a portion of the injection member of the delivery device of FIG. 1 with the injection member in a second position.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “substantially” as used herein does not refer to a particular value, shape, or other configuration, but instead refers to shapes, values, and/or configurations that are readily understood by one of ordinary skill to be similar are analogous to a referenced shape, value, or configuration. For instance, a value that is substantially equal to or similar to another value can be within about 5 percent or within about 10 percent of said value.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

The present disclosure relates to improved delivery devices, systems, and methods. Potential benefits associated with the present disclosure include mitigation of any issues (e.g., unintended clogging of a delivery device and/or clogging of an injection needle) associated with previous injection approaches. As used herein, clogging refers to the partial or complete obstruction of a flow path within a device such as a flow path within a delivery device and/or injection needle. For instance, previous approaches which may require a continuous or near continuous delivery or injection of material such as the simultaneous injection of both a precursor and an accelerant solution to avoid clog formation for form a hydrogel. Otherwise, if a continuous or near continuous injection of the precursor and accelerant solutions is not maintained clogs may develop in the delivery device before a desired volume of material has been delivered to the patient. In such instances, the user may become unable to finalize placement of the material due to the clog formation. Additionally, such approaches may prevent the user from customizing a shape and/or placement location of the material (e.g., hydrogel) but may instead limit the material injection to a given location (e.g., to ensure a continuous or near continuous injection is maintained). As such, a resultant shape of the material may be dictated primarily or entirely by the individual patient anatomy at the given position.

Thus, it may be desirable to permit a user to stop and start an injection or delivery of a material at the given location and permit the user to subsequently start another injection of the remaining material in the delivery device at a different location. Accordingly, the delivery devices and solutions herein are configured to mitigate any clogs (e.g., hydrogel clogs) formed within the delivery device and/or an injection member coupled to the delivery device by sequentially injecting a viscosity-modified buffered precursor solution followed by injection of a buffered accelerant solution to a target site. As such, the delivery devices herein can provide the opportunity for a user to user to stop and start an injection at the given location, and yet at least due to the presence of a viscosity modifier can ensure that the initially injected buffered precursor solution (e.g., a viscosity-modified buffered precursor solution) remains at and thus forms a hydrogel at a target site. As such, the delivery devices and methods herein can permit a user to customize placement or “sculpting” of a material such as hydrogel, and yet can also mitigate issues (e.g., clog formation) that are typically associated with delivery of various materials by delivery devices. While references are made herein to hydrogel delivery devices suitable for hydrogel injection, the present disclosure is not so limited but instead may be employed with various other types of two-part materials such as epoxies, etc.

As mentioned, in some embodiments that delivery devices may be employed with hydrogels. As used herein, a hydrogel refers to a water-containing three-dimensional network of crosslinked polymers. In some embodiments, the injectable hydrogels are shear-thinning and self-assembling injectable hydrogels. The shear-thinning properties of such hydrogels allow for efficient injectability, as the hydrogels exhibit viscous flow under shear. In some embodiments, the injectable hydrogels exhibit yielding behavior. For example, after being subjected to a threshold yield strain, the injectable hydrogels may exhibit sharp decreases in storage and loss moduli, which decreases in moduli are recovered at low strains upon cessation of shear. The self-assembling properties of such hydrogels (also referred to as self-healing properties) allow for re-formation and stabilization of the hydrogel when the shear stress is removed. As used herein, self-assembly and self-healing refer to the spontaneous formation of new bonds within a material after old bonds within the material are broken. The injectable hydrogels of the disclosure may include a carrier fluid. The carrier fluid in the injectable hydrogels may be water. The water may be provided in the form of ultrapure water, water for injection, saline, phosphate buffered saline, or high-ion-content water. In some embodiments, the injectable hydrogels contain between 0.25 weight percent (wt %) or less and 30 wt % or more water, for example, ranging anywhere from 0.25 to 0.5 to 1 to 2.5 to 5 to 10 to 20 to 30 wt %.

In some embodiments, some or all of the components of a delivery device (e.g., a hydrogel delivery device) may be configured to undergo sterilization (e.g., sterilization via steam ultraviolet, gamma radiation, and/or x-ray exposure, etc.). In some embodiments, each of the components of the delivery device 100 may be configured to withstand steam sterilization. For instance, some or all of the components may be formed of materials that are suitable to withstand steam sterilization (e.g., retain their physical form and/or properties during and subsequent to undergoing steam sterilization). Examples of suitable materials (e.g., which retain their physical form) include glass, polycarbonate, polypropylene, rubber, and/or nylon, among other suitable materials.

In various embodiments relating to hydrogels, the injectable hydrogels comprise (a) one or more types of hydrogen bond donors, (b) one or more types of hydrogen bond acceptors, and (c) water. Such hydrogels comprise hydrogen-bond-based crosslinks which dissociate when a shear stress is applied, and which spontaneously self-assemble when the shear stress is removed. Such disassociation may occur, for example, when a shear stress is applied during injection from a syringe. Upon dissociation of the hydrogen-bond-based crosslinks, the hydrogel becomes a viscous liquid that can be transported to a target site though a suitable delivery device, such as a tube (e.g. catheter/microcatheter) or a needle. Once delivered to the target site and the shear stress diminishes, the hydrogen bonds spontaneously re-associate (i.e., self-assemble), reforming the hydrogel at the target site. The transformation of the viscous liquid back into a hydrogel results in improved material retention and mechanical properties.

The injection devices and systems herein may also include a Y-connector that is configured to provide a fluid flow path between each of the first and second reservoirs (e.g., syringes) such that a fluid in the first syringe and a fluid in the second syringe can be injected (e.g., sequentially) into the patient, for example, through a needle or a tube coupled to the Y-connector. For example, the Y-connector may include a first branch lumen having a first end and a second end, a second branch lumen having a first end and a second end, and a common lumen having a first end and a second end. The first end of the first branch lumen and the first end of the second branch lumen may be in fluid communication with the first end of the common lumen at a merge point. The second end of the first branch lumen may terminate at a connector (e.g., a Luer connector) which is configured to connect with a complementary connector of the first syringe barrel, the second end of the second branch lumen may terminate at a connector (e.g., a Luer connector) which is configured to connect with a complementary connector of the second syringe barrel, and the second end of the common lumen may terminate at a connector (e.g., a Luer connector) which is configured to connect with a complementary connector of a needle or a tube (e.g., a Luer connector). While examples of suitable connectors include Luer connectors, the use of other types of connectors is possible.

The injection devices and systems herein may optionally further include various components such as a vial adapter for providing fluid communication between the syringe barrel and the vial. Such a vial adaptor may include a spike, which is configured for the puncturing an elastomeric closure of the vial containing a fluid (e.g., an iodinated polymer composition, a viscosity modifier, etc.), thereby accessing the interior of the vial, and a connector (e.g., Luer connector), which is configured for attachment to the first syringe barrel.

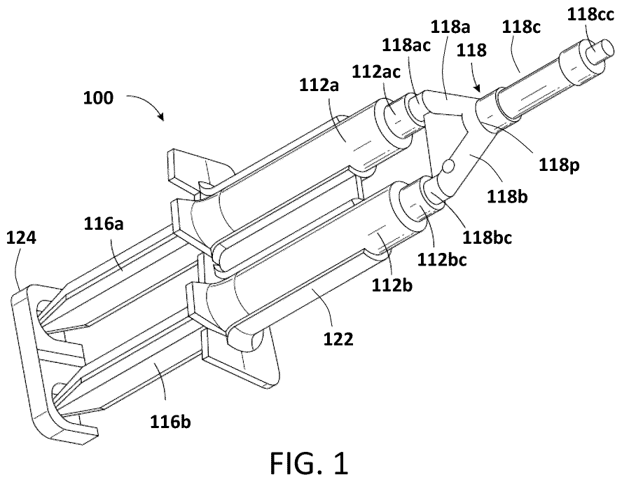

FIG. 1 schematically illustrates an example delivery device 100. The delivery device 100 includes a connector 112ac of the first syringe barrel 112a (e.g., containing a viscosity-modified buffered precursor solution) is connected to a first connector 118ac of a first branch 118a of a Y-connector 118, and a connector 112bc of the second syringe barrel 112b (e.g., containing a buffered accelerant solution) is connected to a second connector 118bc of a second branch 118b of the Y-connector 118.

In some embodiments, a viscosity modifier, as described herein, can be present in a buffered precursor solution thereby forming a viscosity-modified buffered precursor solution contained in the first syringe barrel 112a. Stated differently, the buffered precursor solution and the viscosity modifier can be provided together as a viscosity-modified buffered precursor solution in the same syringe (e.g., in the first syringe barrel 112a). Such embodiments may ensure uniform dispersion of the viscosity modifier throughout the buffered precursor solution and thus ensure a uniform viscosity of the viscosity-modified buffered precursor solution.

However, in some embodiments the first syringe barrel 112a can include a buffered precursor solution (in the absence of a viscosity modifier), and the viscosity modifier can be provided separately (e.g., via a third syringe or via a vial). In such instances, a viscosity of the buffered precursor solution can be modified (by introduction of the viscosity-modifier) prior to, during, and/or subsequent to injection of the buffered precursor solution into target site (e.g., into a body cavity of a patient). Providing the viscosity-modifier separately (separate from the initial buffered precursor solution in the first syringe barrel 118a) can provide an added degree of flexibility to introduce different amounts of viscosity modifier into the buffered precursor solution, and thereby modify the viscosity of the buffered precursor solution to varying degrees, depending for instance on a patient's particular anatomy.

The delivery device 100 includes a first plunger 116a that is movable in the first syringe barrel 112a. The first plunger 116a is movable independent of any movement of a second plunger 116b in the second barrel 112b. Similarly, a second plunger 116b that is movable independent of any movement of the first plunger 116a of the first syringe barrel 112a. In some embodiments, the apparatus 100 may employ a syringe holder 122 which is configured to hold the first and second syringe barrels 112a, 112b, in a fixed relationship. For instance, after the syringe barrels 112a, 112b are attached to the Y-connector, the syringe holder 122 can be attached to the first and second syringe barrels 112a, 112b, as is conventionally known. However, unlike some approaches that may employ a plunger cap (not illustrated) which is configured to hold the first and second plungers 116a, 116b in a fixed relationship (e.g., to permit the first and second plungers 116a, 116b to be pressed at substantially the same time), the delivery device 100 is without (does not include) a plunger cap.

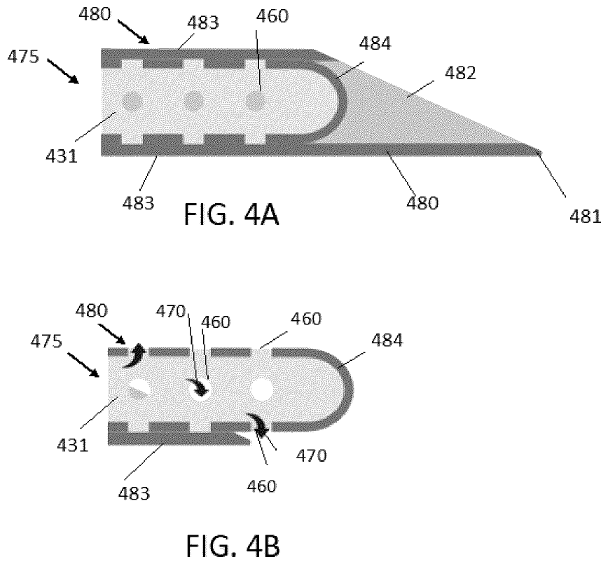

An injection member 119 may be attached to and/or is integral with the common branch connector 118cc, as is conventionally known. The injection member 119 may be manifested as needle or tube. For instance, as illustrated in FIG. 1, the injection member 119 can be manifested as a needle having a beveled or otherwise sharp distal tip. However, in some embodiments, the injection member 119 can be manifested as a tube having a rounded distal tip. In such embodiments, the injection lumen 341 can terminate (end) at a proximal end of the substantially rounded distal tip, as illustrated in FIGS. 4A-4B.

In such embodiments, the injection member 119 can be movably disposed within a lumen of a sheath having a beveled or otherwise sharp distal tip, as detailed herein.

As detailed herein, the injection member 119 can include a plurality of fluid injection ports (not illustrated in FIG. 1). The plurality of fluid ejection ports can be located in a distal end region of the injection member 119 and may be proximate to a distal tip of the fluid ejection member 119. Notably, the plurality of fluid ejection ports can extend through a sidewall of the injection member 119. For instance, each of the plurality of fluid ejection ports can extend from a lumen (internal to the injection member) entirely through a sidewall of the injection member 119 to permit fluid within the lumen to be injected out of the plurality of fluid injection ports to a target site. Having the plurality of fluid injection ports extend through the sidewall of the injection member 119 to permit fluid ejection therethrough can promote aspects herein, such as promoting uniform injection and/or a resultant uniform profile of the injected fluids at a target site.

In some embodiments, a delivery device, system, and/or kit is provided with a viscosity-modified buffered precursor solution (e.g., fluid composition) which comprises: i) a viscosity modifier, as detailed herein, along with ii) a buffered precursor solution including an amino-acid-based polyamine compound and a reactive multi-arm polymer and that is buffered to an acidic pH. For example, the viscosity-modified buffered precursor solution may have a pH ranging, for example, from about 3 to about 6, among other possibilities. In some embodiments, the viscosity-modified buffered precursor fluid solution may contain an acidic buffer that comprise monobasic sodium phosphate, among other possibilities. The viscosity-modified buffered precursor solution may further comprise additional agents, including those described below.

The delivery device, system, and/or kit also includes a buffered accelerant solution (e.g. composition) that comprises a basic buffer. For example, the buffered accelerant solution may contain a basic buffer that provides the buffered accelerant solution with a pH ranging, for example, from about 9 to about 11. In some embodiments, the buffered accelerant solution may comprise sodium borate (e.g., Sodium Tetraborate Decahydrate) and dibasic sodium phosphate, among other possibilities. The buffered accelerant solution may further comprise additional agents, including those described below.

Examples of additional agents for use in the above-described solutions (compositions) include therapeutic agents such anti-angiogenic agents, cytotoxic agents, chemotherapeutic agents, checkpoint inhibitors, immune modulatory cytokines, T-cell agonists, and STING (stimulator of interferon genes) agonists. Examples of additional agents further include imaging agents in addition to any iodine that may be present in the radiopaque products. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope 99mTc, as well as other gamma emitters such as 123I, 125I, 131I, 111In, 57Co, 153Sm, 133Xe, 51Cr, 81mKr, 201Tl, 67Ga, and 75Se, among others, (e) positron emitters, such as 18F, 11C, 13N, 15O, and 68Ga, among others, may be employed to yield functionalized radiotracer coatings, (f) radiocontrast agents, for example, particles of tantalum, tungsten, rhenium, niobium, molybdenum, and their alloys, which metallic particles may be spherical or non-spherical, non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®), and (g) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, the first solution (e.g., a viscosity-modified buffered precursor solution) and the second solution (e.g., a buffered accelerant solution) may independently be provided in various types of reservoirs such as vials, syringes, or other types of reservoirs. For instance, in various embodiments described herein the first solution may be provided via a first syringe and the second solution may be provided via a second syringe that is separate from the first syringe.

In various embodiments, systems and/or kits herein include one or more delivery devices for delivering the compositions described herein to a subject. For instance, the viscosity-modified buffered precursor solution that is buffered to an acidic pH and comprises the amino-acid-based polyamine compound and the reactive multi-arm polymer and the buffered accelerant solution that is buffered to basic pH may be combined sequentially, as detailed herein, to form covalently crosslinked hydrogels in vivo.

As used herein, a viscosity modifier refers to a substance that increases a viscosity of a solution, namely a buffered precursor solution that is suitable for formation of a hydrogel. For instance, the viscosity modifier may alter the arrangement of and/or increase interactions between molecules within the buffered precursor solution thereby increasing the viscosity of the buffered precursor solution (e.g., as compared to a viscosity of the buffered precursor solution alone under the same conditions such as the same temperature). That is, the presence of the viscosity modifier in the amounts described herein can yield an increase in viscosity (relative to the initial unmodified viscosity) of at least five percent, at least 10 percent, at least 15 percent, at least 20 percent, or at least 30 percent, as detailed herein. This increase in viscosity can promote aspects herein such as ensuring the viscosity-modified precursor solution remains at an injection location in a target site, as compared to use of the lower viscosity precursor alone (e.g., which may be prone to undesired flow or movement away from a location at the target site).

Examples of suitable viscosity modifiers to increase a viscosity of a buffered precursor solution include polylactic acid-co-glycolic acid (PLGA) and Pegylated PLGA including diblock (PEG-PLGA) or triblock (PEG-PLGA-PEG or PLGA-PEG-PLGA) or multiblock, etc., polyvinyl alcohol (PVP), polylactic acid (PLA), and hydrophilic polymers. Examples of suitable hydrophilic polymers include natural hydrophilic polymers include polysaccharides (e.g., dextran, alginate, chitosan, agarose, and pullulan) and proteins (e.g., albumin, gelatin, collagen, lectin, legumine, and vicilin). In some embodiments, the viscosity modifier can be selected from a group consisting of PLGA, PVP, PLA and hydrophilic polymers. In some embodiments, the viscosity modifier can be selected from a group consisting of PLGA, Pegylated PLGA, PVP, and PLA.

In some embodiments, the viscosity modifier is present at a weight ratio of about 0.5 to about 10 based on a total weight of the viscosity-modified buffered precursor solution. All individual values and sub-ranges from about 0.5 to about 10 are included.

In some embodiments, the buffered precursor solution is present at a weight ratio of about 90 to about 99.5 weight percent based on a total weight of the viscosity-modified buffered precursor solution. All individual values and sub-ranges from about 90 to about 99.5 weight percent are included.

In some embodiments, the viscosity modifier and the buffered precursor solution can together from at least 90 weight percent, at least 95 weight percent, at least 97 weight percent, at least 98 weight percent, at least 99 weight percent, or 100 weight percent of a total weight of the viscosity-modified buffered precursor solution.

In some embodiments, wherein the viscosity-modified buffered precursor solution has a viscosity that is at least 5 percent higher than a viscosity of the buffered precursor solution. For instance, the viscosity-modified buffered precursor solution can have a viscosity that is at least 5 percent, at least 10 percent, at least 15 percent, at least 20 percent, or at least 30 percent higher than a viscosity of the buffered precursor solution (without the addition of the viscosity modifier). As mentioned, increasing the viscosity of the buffered precursor solution by the addition of the viscosity modifier to form the viscosity-modified buffered precursor solution can ensure that the viscosity-modified buffered precursor solution remains at a target site (e.g., injection between the rectum and the prostate for spacing in radiation therapy for prostate cancer), and thus can enhance the efficacy of the spacing provided by a resultant hydrogel formed from the viscosity-modified buffered precursor solution and a buffered accelerant solution in radiation therapy for rectal cancer.

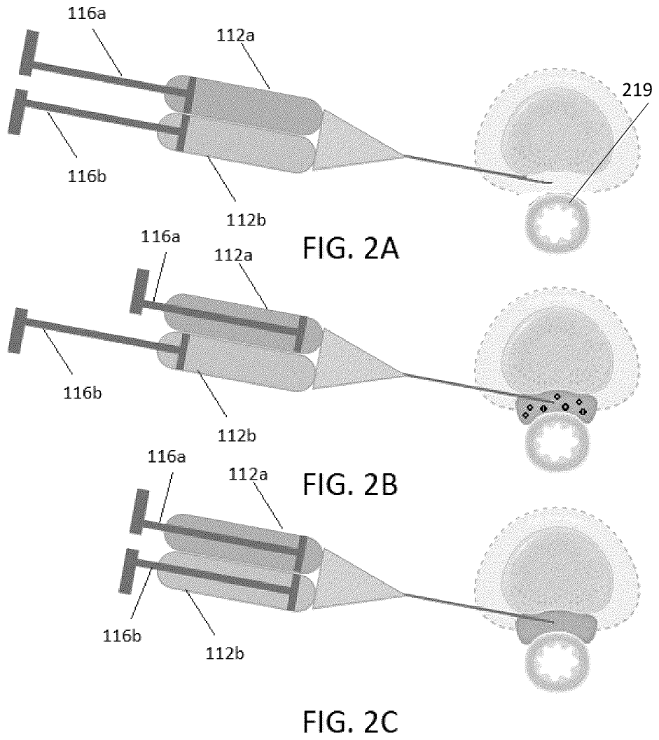

FIGS. 2A-2C illustrate simplified views of a target site 219 (e.g., between the rectum and the prostate for spacing in radiation therapy for prostate cancer), and the delivery device 100 in different positions that are suitable for the sequential injection of fluids to form a hydrogel. During operation, the first plunger 116a is pressed into the first syringe barrel 112a, forcing at least a portion of the first solution (e.g., viscosity modified buffered precursor solution) through the lumen of the first branch 118a of the Y-connector 118 into the injection member 119 and thus to be injected via the injection member 119 at a target site 219.

For example, FIG. 2A illustrates the first plunger 116a and the second plunger 116b both being located at an initial position (e.g., in which the first plunger 116a and the second plunger 116b have not been pressed). In this position, the first solution (fluid) remains in the first syringe barrel 112a and the second solution (fluid) remains in the second syringe barrel 112b. Stated, differently, no fluid is injected when the first plunger 116a and the second plunger 116b both being located at an initial position. However, FIG. 2B illustrates the first plunger 116a in a second position in which the first plunger 116a has been pressed into the first syringe barrel 112a, forcing at least a portion of the first solution (e.g., the viscosity-modified buffered precursor solution, where the black dots represent the presence of the viscosity modifier) through the lumen of the first branch of the Y-connector 118 and through the injection member 119 to the target site 219.

As mentioned, in some instances, injection of the first solution can be started and stopped. For instance, a portion of the first solution can be injected at a first location in a target site 219, the injection device 100 (e.g., the injection member 119) can repositioned at a second (different) location, and some or all of a remaining portion of the first solution can be injected at the second location in the target site 219.

Subsequent to injection of some or all of the first solution, the second plunger 116b is pressed into the second syringe barrel 112b, forcing a second solution (e.g., the buffered accelerant solution) through lumen of the second branch 118b of the Y-connector, as illustrated in FIG. 2C. Moving the second plunger 116b from the first position to the second position (independent of a position of the first plunger 116a) forces the second solution (e.g., the buffered accelerant solution) through the lumen of the second branch of the Y-connector 118 and the injection member 119 to the target site 219. The first solution (e.g., the buffered precursor solution) and the second solution (e.g., the buffered accelerant solution) thus meet and mix predominantly in vivo at the target site 219 (e.g., rather than at a merge point 118p within a lumen of the apparatus 100 as is the case in some previous approaches). For instance, combining a buffered accelerant solution with the viscosity-modified buffered precursor solution in situ can increase the pH of the resulting mixture, causing crosslinking e.g., between an iodinated polymer and the polyamine, which leads to the formation of a hydrogel at the target site 219.

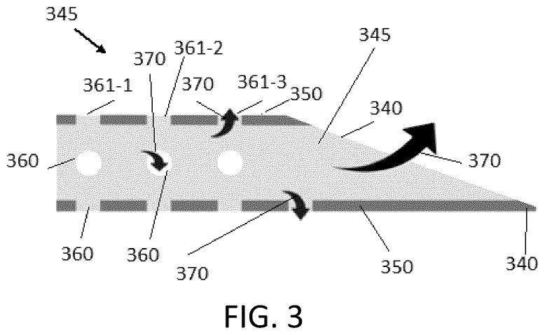

FIG. 3 illustrates an enlarged view of a portion 345 (corresponding to element 127 in FIG. 1) of the injection member 119 of FIG. 1. As illustrated in FIG. 3, the portion of 345 of the injection member corresponds to a distal end region 345 of the injection member. The distal end region terminates in the distal tip 340. The injection member can have an injection lumen 341 extending from a proximal end to the distal tip 340 of the injection member. That is, the injection lumen can extend substantially longitudinally therethrough and that is in fluid communication with a common lumen in the Y-connector.

The injection lumen 341 can be in fluid communication with an opening 345 in the distal tip 340 and/or can be in fluid communication with a plurality of a plurality of fluid injection ports 360. In some embodiments, the injection lumen 341 can be in direct fluid communication with the opening 345 in the distal tip 340 and/or the plurality of a plurality of fluid injection ports 360. For instance, as illustrated in FIG. 3, the injection lumen 341 can be in direct fluid communication (with an absence of an intervening member) with an opening 345 in the distal tip 340 and each fluid injection port of the plurality of a plurality of fluid injection ports 360. For instance, the injection lumen 341 can extend through the opening 345 in the distal tip 340, as illustrated in FIG. 3. However, in some embodiments the injection lumen 341 can in direct fluid communication with only the plurality of fluid injection ports 360 (e.g., when the distal tip is a substantially rounded continuous distal tip which has an absence of an opening therein), a described in more detail with respect to FIGS. 4A-4C.

In any case, the plurality of fluid injection ports 360 can extend through (entirely through) a portion of a sidewall 350 of the injection member and thus can permit a fluid to be injected therethrough. For instance, the plurality of fluid injection ports 360 can be configured to permit the sequential injection of a first solution (e.g., a viscosity-modified buffered precursor solution) and subsequently a second solution (e.g., a buffered accelerant solution which is without a viscosity modifier), as described herein. For instance, the plurality of fluid injection ports 360 can be shaped, sized, and/or spaced along the injection member to promote aspects herein such as promoting the sequential injection of a first solution via at least the plurality of fluid injection portions 360 and subsequently the injection of the second solution via at least the plurality of fluid injection ports 360.

For instance, in some embodiments each fluid ejection port of the plurality of fluid injection ports 360 can be substantially the same shape and/or the same size. For example, in some embodiments each fluid ejection port of the plurality of fluid injection ports 360 can be substantially the same shape. In some embodiments each fluid ejection port of the plurality of injection ports 360 can be substantially the same size (e.g., can have the same diameter). In some embodiments, in some embodiments each fluid ejection port of the plurality of fluid injection ports 360 can be substantially the same shape and size. Having the plurality of injection ports 360 be substantially the same shape and size can promote aspects herein such a promoting the uniform injection of a solution therethrough. For instance, in some embodiments, each of the plurality of fluid injection ports 360 can be substantially circular fluid ejection port having substantially the same diameter. However, other shapes or configurations are possible. For example, in some embodiments the fluid injection ports 360 can vary in size or shape. For instance, the injection ports may vary in size and/or shape along a longitudinal axis of the injection member 119.

In some embodiments, the plurality of fluid injection ports 360 can be configured as a plurality of sets 361-1, 361-2, and 361-3 of fluid injection ports (collectively referred to as “sets 361”). Each set of the plurality of sets of fluid injection ports 361 can spaced apart along a longitudinal axis of the fluid injection member of the delivery device (e.g., the fluid injection device), as illustrated in FIG. 3. Each set of the fluid ejection ports includes a plurality of fluid injection ports 360. For example, each set can include two, three, four, five, or six fluid ejection ports, among other possibilities. For instance, each set can include a plurality of fluid injection ports 360 extending circumferentially about an annular sidewall 350 of the injection member, as represented in FIG. 3. In such instances, the plurality of fluid ejection ports can be equally spaced about the circumference of the annular sidewall 350 and can be located substantially the same distance from the distal tip 340 of the fluid injection member, as illustrated in FIG. 3. For instance, each of the plurality of fluid ejection ports 360 can be configured as diametrically opposed pairs located in opposing portions of the annular sidewall 350. For instance, a set of fluid ejection ports can include a first pair of fluid ejection ports (e.g., formed of a first fluid ejection port 360-1 and a second fluid ejection port 360-2) can be located at a “top” portion and a “bottom” portion of the annular sidewall, as illustrated in FIG. 3). Similarly, the set can include a second pair of fluid ejection ports include a third fluid ejection port 360-3 located on a “first side portion” located between the “top” and the “bottom portion” and a fourth fluid ejection port (not illustrated) located on “second side portion” which opposes the first side portion. However, the injection member can include any arrangement and/or quantity of sets and/or fluid ejection ports.

In some embodiments, each fluid ejection port of the plurality of fluid ejection ports 360 can have a diameter that is configured to permit the viscosity modifier to pass therethrough. That is, the diameter of the plurality of fluid ejection ports can be at least 3%, at least 5%, at least 10%, at least 20%, at least 30%, at least 40% or at least 50% larger than a diameter of a viscosity modifier (e.g., a viscosity modifier particle), among other possible values. Having the plurality of fluid ejection ports 360 have a diameter or size that is larger than the viscosity modifier can permit the viscosity modifier (e.g., disposed within the viscosity-modified buffered precursor solution) to readily pass therethrough. For example, the diameter of each of the fluid ejection ports 360 can be in a range from about 10 μm to about 1000 μm or from about 1 μm to about 2000 μm, among other possible values. All individual values and sub-ranges from about 1 μm to about 2000 μm are included.

FIG. 4A illustrates an enlarged view of a portion 475 of the injection member of the delivery device of FIG. 1 with the portion 475 of the injection member in a first position relative to a sheath 480. Similarly, FIG. 4B illustrates an enlarged view of the portion 475 of the injection member of the delivery device of FIG. 1 with the portion 475 of the injection member in a second position relative to the sheath 480.

The sheath 480 can be formed of a continuous solid material (e.g., without any apertures extending through a sidewall thereof). The sheath 480 can terminate in a distal tip 482. In some embodiments, the sheath 480 can include a sidewall 483 defining a lumen 482. For instance, the sidewall 483 can be a continuous sidewall without an opening extending therethrough, as illustrated in FIGS. 4A-4B.

As illustrated in FIGS. 4A-4B, the lumen 482 can extend substantially longitudinally through the sheath 480. For instance, the lumen 482 can extend substantially longitudinally through an entire length or at least a distal end region of the sheath 480. In some embodiments, the lumen 482 can extend at least through the distal tip 482 of the sheath 480. In some instances, the lumen 482 can extend through the distal tip 482 of the sheath 480 and can extend through a proximal end or tip (not illustrated) of the sheath 480. In any case, the lumen 482 can be configured to receive at least the portion 475 of the injection member therein. In some instances, the portion 475 of the injection member can be friction fit within the lumen 482. For instance, the sheath 480 can be manifested as an annular sheath defining an annular lumen that is configured to receive and friction fit with at least the portion 475 of the injection member therein.

In some instances, the sheath 480 can have a length (taken along a longitudinal axis of the portion 475 of the injection member) that is configured to permit a distal tip 484 of the portion 475 of the injection member to be selectively exposed (e.g., protrude a distance distally from a distal tip 481 of the sheath 480). For instance, as illustrated in FIG. 4A, the portion 475 of the injection member is disposed entirely within the sheath 480. In this configuration, each of the fluid injection ports 460 is overlaid and blocked by the sheath 480. As such, injection of the fluids is not permitted, yet such as configuration may be suitable during initial navigation to or placement of the portion 475 of the injection member at a target site. For instance, the distal tip 681 of the sheath can be beveled or an otherwise sharpened distal tip can be suitable for navigating tissue.

However, the portion 475 of the injection member can be configured to be movably disposed within the lumen 482 (e.g., is configured to translate longitudinally relative to at least the distal tip 482 of the sheath 480 to expose at least a distal tip 484 of the portion 475 of the injection member. For example, some or all of the fluid injection ports 460 can be exposed (e.g., are no longer overlaid and blocked by the sidewalls of the sheath) in this second (exposed) configuration. As such, injection of the fluids herein can be permitted when in the second configuration. Additionally, in this configuration the distal tip 481 can be recessed (is positioned proximal to the distal tip 484) of the portion 475 of the injection member. Having the distal tip 481 (e.g., a bevel or otherwise sharpened distal tip) be recessed relative to the distal tip 484 can mitigate any inadvertent tissue damage that may otherwise be imparted by the distal tip 484 (e.g., once the portion 475 of the injection member is physically located at the target site). For example, the distal tip 484 can be a substantially rounded distal tip, as illustrated in FIGS. 4A-4B. Employing the substantially rounded distal tip 484 can further mitigate any unwanted tissue damage. In some instances, the distal tip 484 can be closed (e.g., the injection lumen 441 extending through the portion 475 of the injection member does not extend through the distal tip 484. In such instances, fluid provided via the injection lumen 441 can be injected via the plurality of fluid injection ports 460 (e.g., which are analogous to or similar to the fluid injection ports 460), as described herein. As mentioned, the delivery devices (e.g., hydrogel delivery devices) herein can perform an initial material (e.g., hydrogel) injection or delivery, and can subsequently pause and restart material injection or delivery a plurality of times (e.g., two) times, as compared to other existing approaches the require continuous or near continuous material injection or delivery (e.g., at a fixed location).

The material injection systems and kits herein can include a delivery device. However, in some embodiments one or more additional components (e.g., sensing components, sterile water reservoirs, viscosity modifier reservoirs, etc. can be provided).

Methods of material (e.g., hydrogel) delivery or injection are described herein. The methods can be employed with the injection devices described herein. For instance, in some embodiments, the method can further include sequentially pressing a first plunger into a first syringe barrel to cause a portion (some or all) of a first solution in the first syringe to be injected to a target site. For example, a method can include pressing a first plunger into a first syringe barrel of a first syringe to cause a portion of a viscosity-modified buffered precursor solution for formation of a hydrogel in the first syringe to be injected at a first position in a body cavity via at least a plurality of injection ports in an injection member coupled to the first plunger.

Notably, the methods herein can permit stopping and starting the injection of material (e.g., starting and stopping injection of a viscosity-modified precursor solution). For instance, the method can include pausing pressing the first plunger (e.g., subsequent to initially pressing the first plunger to cause injection of a portion of a first solution to be injected at the first location in the target site). For instance, the methods herein can include pausing the injection by pausing pressing first plunger (e.g., pausing an application of pressure to the first plunger subsequent to injection the portion of the first solution (e.g., prior to injection of all of the first solution). Subsequent to pausing the injection, the methods herein can include altering a position of an injection member. For instance, the injection member can be moved from a first location at the target site to a second (different) location at the target site. Subsequently, the methods herein can include resuming the injection by resuming pressing (e.g., resuming the application of pressure) to the first plunger to cause at least some of a remaining portion of the first solution to be injected at the second location at a target site.

The methods herein can include subsequently injecting a buffered accelerant solution. For instance, the buffered accelerant solution can be injection subsequent to at least the initial injection of the viscosity-modified buffered precursor solution at the first site. For example, the method may include subsequent to injecting the viscosity-modified buffered precursor solution at the second position, pressing a second plunger into a second syringe barrel of a second syringe to cause a buffered accelerant solution in the second syringe to be injected in the body cavity via at least the plurality of injection ports.

In some embodiments, the methods herein can include flushing the injection member with a rinsing solution subsequent to injection of the first solution (e.g., at one or more locations at a target site) to remove any residual amount of the first solution that may remain within a lumen of the Y-connector and/or in the injection lumen of the injection member prior to injection of a second solution. For instance, the first syringe can be decoupled from the Y-connector, a third syringe containing a rinsing fluid can be coupled to the Y-connector (where the first syringe was previously coupled), and a third plunger of the third syringe can be pressed to flush the injection device, and namely the Y-connector and the injection member with the rinsing solution prior to injection of the second solution (e.g., the buffered accelerant solution). The rinsing fluid may be provided in the form of ultrapure water, water for injection, saline, phosphate buffered saline, or high-ion-content water, among other types of fluids such as anesthetics fluids, etc. For instance, the rinsing fluid may be saline. Flushing the injection device (e.g., the Y-connector and/or the injection member) subsequent to the injection of the first solution and prior to injection of any of the second solution can promote aspects herein, such as mitigating any unintended hydrogel clog formation within the injection member and/or the Y-connector. However, in some embodiments the same injection member can be utilized (e.g., without replacement and without flushing) to inject the first solution and to subsequently inject the second solution, as detailed herein.

In some embodiments, a method of hydrogel injection can comprise connecting a first syringe barrel of a first syringe to a first branch lumen of the Y-connector; connecting a second syringe barrel of the second syringe to a second branch lumen of the Y-connector; connecting an injection member (e.g., a needle or tube such as a catheter tube or cannula) to the common lumen of the Y-connector to form a delivery device. The methods herein can further include sequentially pressing the first plunger and the second plunger into the first syringe barrel and the second syringe barrel, respectively, to cause a portion of first solution in the first syringe to be injected to a target site prior to a second solution in the second syringe being injected to the target site to form a hydrogel at the target site. That is, the first solution and the second solution can mix in vivo at a target site to form hydrogel, as detailed herein. Notably, the methods herein can permit stopping and starting the injection of one or more fluids for formation of a hydrogel, as detailed herein.

The systems and kits described herein may be used for in a variety of medical procedures and/or non-medical procedures. For example, the systems and kits of the present disclosure may be used to provide fiducial markers, to provide tissue augmentation or regeneration, to provide a filler or replacement for soft tissue, to provide mechanical support for compromised tissue, to provide a scaffold, as a carrier of therapeutic agents in the treatment of diseases and cancers and the repair and regeneration of tissue, among other uses.

The systems and kits of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to implant a therapeutic-agent-releasing depot comprising a hydrogel, a tissue augmentation procedure comprising implanting a hydrogel, a procedure to introduce a hydrogel between a first tissue and a second tissue to space the first tissue from the second tissue.

The systems and kits of the present disclosure may be used in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. A delivery device comprising:
a first syringe comprising a first plunger and a first syringe barrel that terminates in a first connector;
a second syringe comprising a second plunger and a second syringe barrel that terminates in a second connector;
a Y-connector coupled to the first syringe and the second syringe; and
an injection member coupled to the Y-connector, wherein the injection member includes a plurality of fluid injection ports that extends through a sidewall of the injection member.

2. The delivery device of claim 1, wherein the injection member is a needle or a tube.

3. The delivery device of claim 1, wherein the plurality of fluid injection ports is located at a distal end region of the injection member.

4. The delivery device of claim 1, wherein the injection member includes an injection lumen extending substantially longitudinally therethrough and that is in fluid communication with a common lumen in the Y-connector, wherein the injection lumen is in fluid communication with at least the plurality of fluid injection ports.

5. The delivery device of claim 4, wherein a distal tip of the injection member is beveled.

6. The delivery device of claim 4, wherein:
a distal tip of the injection member is substantially rounded; and
the injection member is movably disposed within a lumen of a sheath, the sheath having a beveled distal tip.

7. The delivery device of claim 6, wherein the injection lumen terminates at a proximal end of the substantially rounded distal tip.

8. The delivery device of claim 1, wherein each fluid ejection port of the plurality of injection ports is substantially the same shape and the same size.

9. The delivery device of claim 1, wherein each fluid ejection port of the plurality of fluid ejection ports is a substantially circular fluid ejection port.

10. The delivery device of claim 1, wherein the sidewall is an annular sidewall, and wherein the plurality of fluid injection ports is located circumferentially about the annular sidewall.

11. The delivery device of claim 1, wherein the plurality of fluid injection ports is located in a distal end region of the injection member.

12. The delivery device of claim 1, wherein:
the first plunger is movable between a first position and a second position independent of a position of the second plunger; and
the second plunger is movable between a first position and a second position independent of a position of the first plunger.

13. The delivery device of claim 1, wherein the plurality of fluid injection ports is configured as a plurality of sets of fluid injection ports that are spaced apart along a longitudinal axis of the delivery device, and wherein each set of fluid injection ports includes a plurality of fluid injection ports extending circumferentially about the annular sidewall of the injection member.

14. The delivery device of claim 1, wherein:
wherein the first syringe contains a first solution comprising a viscosity-modified buffered precursor solution including a buffered precursor solution and a viscosity modifier; and
the second syringe contains a second solution comprising a buffered accelerant solution.

15. The delivery device of claim 14, wherein each fluid ejection port has a diameter that is configured to permit the viscosity modifier to pass therethrough, and wherein the viscosity modifier comprises polylactic acid-co-glycolic acid (PLGA), polylactic acid (PLA), or a hydrophilic polymer.

16. A delivery device comprising:
a first syringe comprising a first plunger and a first syringe barrel that terminates in a first connector, wherein the first syringe contains a first composition comprising a viscosity-modified buffered precursor solution including a buffered precursor solution and a viscosity modifier; and,
a second syringe comprising a second plunger and a second syringe barrel that terminates in a second connector, wherein the second syringe contains a buffered accelerant solution;
a Y-connector coupled to the first syringe and the second syringe; and
an injection member coupled to the Y-connector, wherein the injection member includes:
an annular sidewall with an injection lumen extending therethrough; and
a plurality of fluid injection ports that are in fluid communication with the injection lumen and that extend through the annular sidewall; and
wherein the delivery device is configured to sequentially inject the viscosity-modified buffered precursor solution followed by the buffered accelerant solution.

17. The delivery device of claim 16, wherein the viscosity modifier comprises polylactic acid-co-glycolic acid (PLGA), polylactic acid (PLA), or a hydrophilic polymer.

18. The delivery device of claim 16, wherein the viscosity modifier is present in an amount in a range from about 0.5 to about 10 weight percent based on a total weight of the viscosity-modified buffered precursor solution.

19. The delivery device of claim 16, wherein the viscosity-modified buffered precursor solution has a viscosity that is at least five percent higher than a viscosity of the buffered precursor solution.

20. A kit comprising:
a first reservoir containing a first composition comprising a viscosity-modified buffered precursor solution including a buffered precursor solution and a viscosity modifier;
a second reservoir containing a second composition comprising a buffered accelerant solution; and
a delivery device comprising:
a Y-connector coupled to the first reservoir and the second reservoir; and
an injection member coupled to the Y-connector, wherein the injection member includes:
an annular sidewall with an injection lumen extending therethrough; and
a plurality of fluid injection ports that are in fluid communication with the injection lumen and that extend through the annular sidewall; and
wherein the delivery device is configured to sequentially inject the viscosity-modified buffered precursor solution followed by the buffered accelerant solution.
